(19) United States
(12) Patent Application Publication
(10) Pub. No.: US 2021/0128341 A1
(43) Pub. Date: May 6, 2021

(54) THE DRIP CATCHER

(71) Applicant: William Wayne, Bellevue, WA (US)

(72) Inventor: William Wayne, Bellevue, WA (US)

(21) Appl. No.: 16/949,539

(22) Filed: Nov. 2, 2020

Related U.S. Application Data
(60) Provisional application No. 62/973,880, filed on Oct. 31, 2019.

(51) Int. Cl. A61F 5/44

(52) U.S. Cl. CPC A61F 5/4404

(57) ABSTRACT

The Drip Catcher (100) for a male user has walls (101) to prevent urine from splashing, a drain (103) where urine flows down into the toilet (109), handles (102) plus an alternate handle (112) in the back of the device. The bowl attachment holder (106) secures the device to the toilet bowl.

### CROSS REFERENCE TO RELATED APPLICATION

This application claims the benefit, under 35 U.S.C. § 119(e), of U.S. Provisional Application No. 62/973,880, filed Oct. 31, 2019, the disclosure of which is incorporated herein by reference in its entirety.

### FEDERALLY SPONSORED RESEARCH OR DEVELOPMENT

Not Applicable.

### BACKGROUND

My invention attempts to keep urine off the toilet floors. Why? Because urine smells, is a mess to clean and belongs in the toilet bowl. I heard about a friend who has her husband sit while urinating. Another friend made a poster for his guest toilet that read: “Please sit to urinate—I do not want to clean up your urine drips that fall to the floor.” Now, my wife recently said she wants me to sit while urinating as well. I don't like that idea. I thought about using a funnel, like we used for our farm tractor, but it didn't seem practical. I go to the bathroom about four times during the day and countless more at night. I told my wife, men can't shut off like a light switch. We are more like an old drippy faucett. So I invented the DRIP CATCHER to catch urine drips from hitting the floor. This has been the solution to this problem. It catches the drops and they flow down into the bowl. One may think the DRIP CATCHER is just a niche for older males, however, females in particular are loving the fact that is is keeping their family's urine off the their bathroom floor.

### SUMMARY

The DRIP CATCHER extends 3 inches in front of the toilet bowl and catches drips that would fall to the floor. It fits over the front rim and is easy to put on and lift off. THE DRIP CATCHER is set at an angle so that the urine flows down into the bowl. A male usually stands 3 inches from the toilet bowl rim so their pants do not touch the toilet bowl. Some males stand even further back, especially if wearing shorts. Men who are six feet tall and taller have a further drop to the bowl making it very challenging for all their urine to fall inside the toilet. While male senior citizens also have a tough time getting their urine into the toilet, as it is very challenging for them to control the direction of their urine. The DRIP CATCHER helps to solve all these problems. The DRIP CATCHER has handles on each side and a longer handle can be attached for easy carrying. To clean the DRIP CATCHER, use a spray bottle containing water and a small amount of soap or bleach. A cleaning brush is included. The DRIP CATCHER can be stored in a decorated cardboard box suitable for placement on the toilet water tank.

### DETAILED DESCRIPTION OF THE INVENTION

The DRIP CATCHER is unique as we researched the (PTAC) database and did not find anything similar on file. When placed on the toilet bowl it will prevent urine from falling onto the floor.

It is made up of unbreakable plastic and has two handles for easy carrying. The walls of the device keep urine from splashing and allow it to flow downward through the drain of the device. The device extends three inches past the toilet bowl and catches the urine as it flows down the bowl. The device is 5′ in width from handle to handle and 5″ in length from the back wall to the beginning of the drain. The device can be cleaned easily as urine is washable from the unbreakable plastic with a spray bottle containing water with a small amount of soap or bleach and a cleaning brush.

The DRIP CATCHER fits in a white card board box with the dimensions of 12″×6″4″. This may be easily placed on top of the toilet water tank. Directions are listed on the inside cover.

### DESCRIPTION OF THE FIGURES

The DRIP CATCHER device 100 for a male user.

FIG. 1 is a perspective view of an embodiment of the device according to the invention.

101 is the wall keeping the urine from splashing outside the device. 102 is the handle for use in carrying the device. 103 is the drain that empties urine into the toilet bowl. 104 is the width 5″ wide across from handle to handle. 105 is the length 5″ long from the back wall to the start of the drain.

FIG. 2 is an underneath view of the embodiment of the invention. 101 is the wall keeping the urine from splashing outside the device. 102 is the handle for use in carrying the device. 103 is the drain that empties urine into the toilet bowl. 106 is the bowl attachment holder that connects to the front of the toilet bowl.

FIG. 3 is a perspective view above the embodiment of the invention. 101 is the wall keeping the urine from splashing outside the device. 102 is the handle for use in carrying the device. 103 is the drain that empties urine into the toilet bowl.

FIG. 4 is a side angle view of the embodiment of the invention. 101 is the wall keeping the urine from splashing outside the device. 102 is the handle for use in carrying the device. 103 is the drain that empties urine into the toilet bowl. 106 is the bowl attachment holder that connects to the front of the toilet bowl. 107 is the drain angle that drains the urine to the toilet bowl. 108 is the angle of the device above the toilet bowl allowing urine to flow downward.

FIG. 5 is a high angle perspective view of the device attached to a toilet. 109 is the toilet bowl.

FIG. 6 is a side view of the device attached to the toilet. 101 is the wall keeping the urine from splashing outside the device. 102 is the handle for use in carrying the device. 103 is the drain that empties urine into the toilet bowl. 106 is the bowl attachment holder that connects to the front of the toilet bowl. 109 is the toilet bowl.

FIG. 7 is an underneath angle view of the device showing the bolt in the holder that attaches to a urinal. 101 is the wall keeping the urine from splashing outside the device. 102 is the handle for use in carrying the device. 103 is the drain that empties urine into the toilet bowl. 106 is the bowl attachment holder that connects to the front of the toilet bowl. 110 is the bolt inside the holder which secures tightly when attached to a urinal.

FIG. 8 is the side view of the device showing the nut and bolt attached to the holder.

101 is the wall keeping the urine from splashing outside the device. 102 is the handle for use in carrying the device. 103 is the drain that empties urine into the toilet bowl. 106 is the bowl attachment holder that connects to the front of the toilet bowl. 110 is the bolt inside the holder which secures tightly when attached to a urinal.

FIG. 9 is the angle perspective view of the device with the removable handle. 101 is the wall keeping the urine from splashing outside the device. 102 is the handle for use in carrying the device. 103 is the drain that empties urine into the toilet bowl. 111 is the small hole in the back wall of the device the handle can screw into. 112 is the handle that screws into the small hole in the back wall of the device.

FIG. 10 is the perspective view of the box containing the embodiment of the device according to the invention. 113 is the white box with the dimensions of 12″×6″×4″.

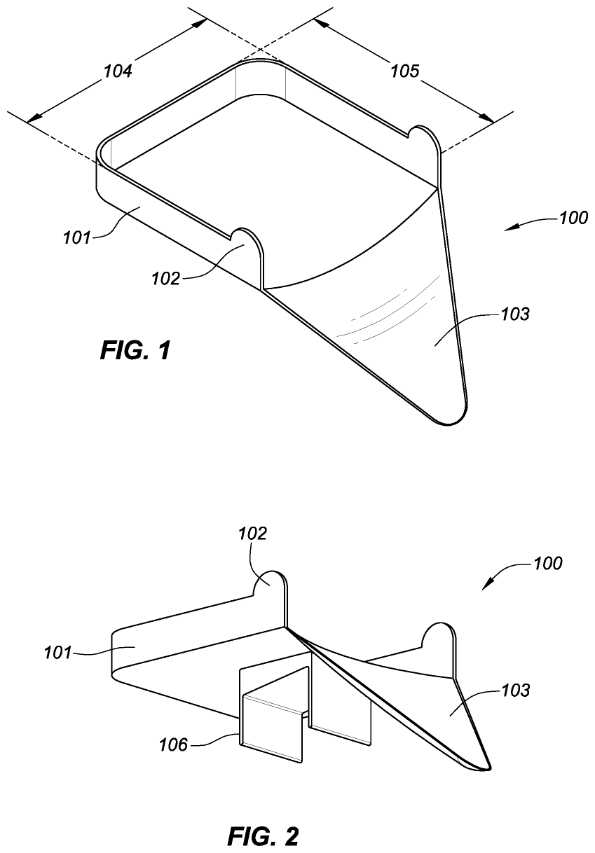

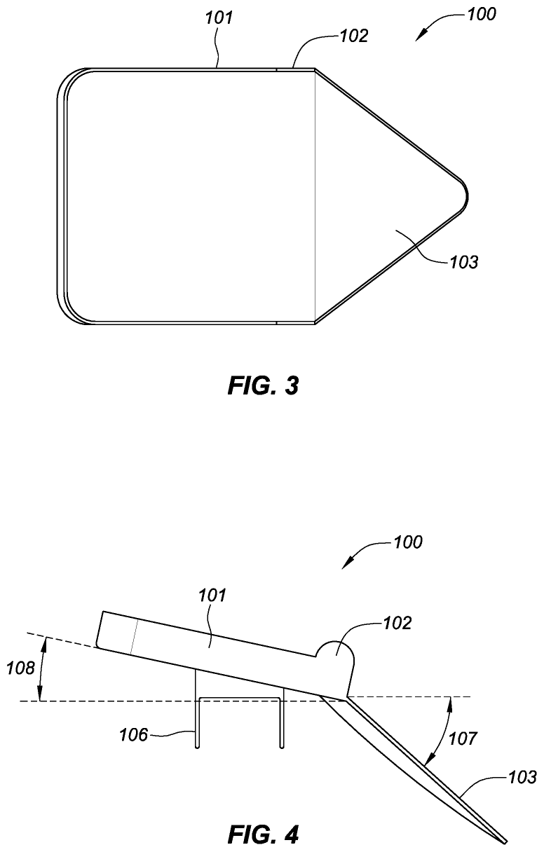

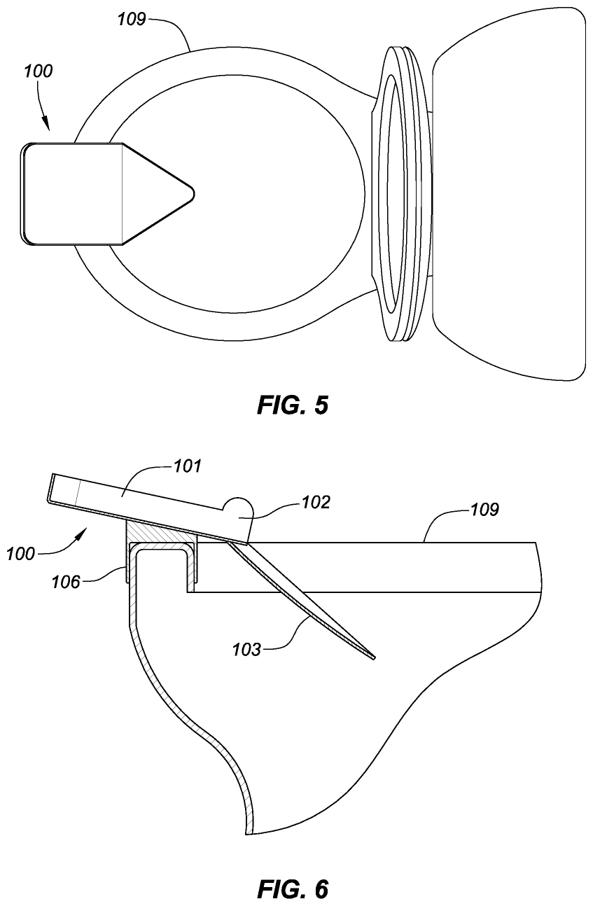

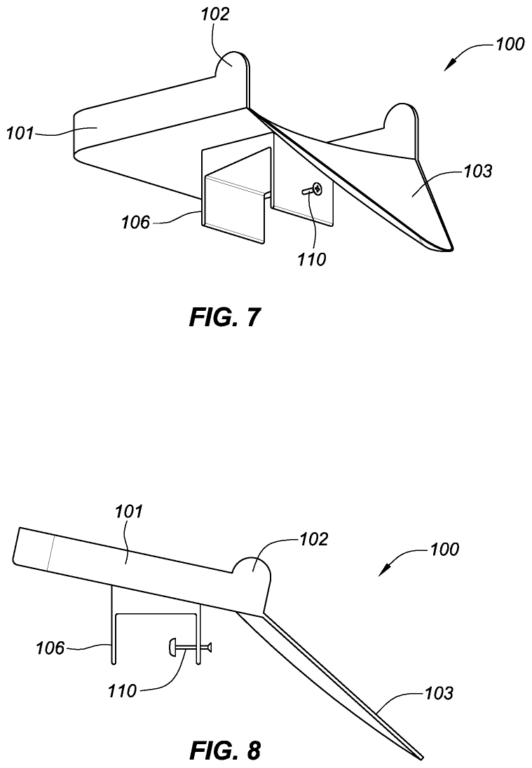

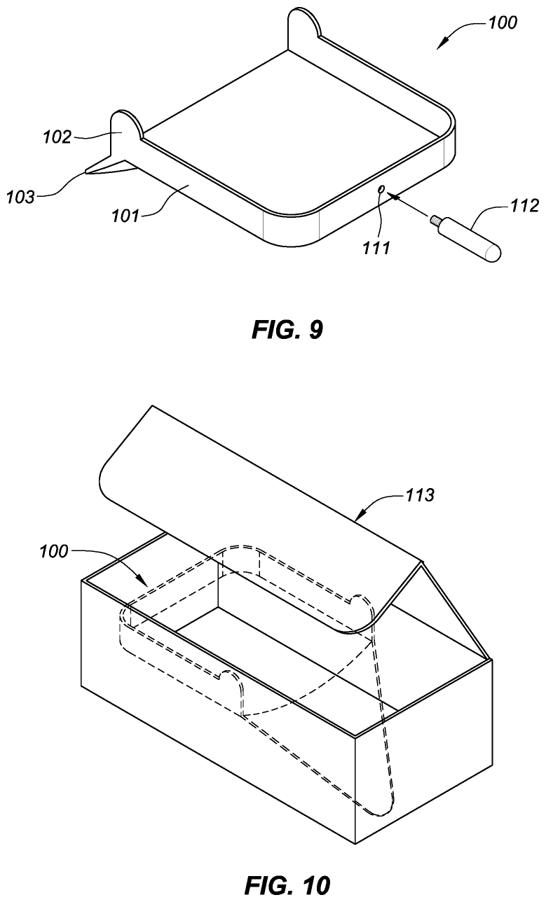

What is claimed is:

1. The Drip Catcher is made of hard unbreakable plastic.
a) It has no movable parts except for the handle.
b) It extends three inches in front of the bowl so that it catches drips.
c) It is placed on a slant, on the bowl's rim, so the urine flows down to the bottom.
d) It has permanent handles on each side for easy carrying.
e) Underneath the Drip Catcher is the holder of the Drip Catcher, which fits on the toilet bowl. (see FIG. 6)
f) It can be cleared with a spray bottle of water containing a small amount of soap or bleach.

2. The Drip Catcher can also be placed on a urinal.
a) It can be secured to a urinal with a nut and bolt 110 on FIG. 8.

3. The Box to the Drip Catcher fits on top of the toilet water tank.
a) The box is white and splash proof.
b) It contains the Drip Catcher and removable handle.
c) Directions are posted on the inside of the box's lid.
d) The box size is 12″×6″×4″ (see FIG. 10).
